(19) United States
(12) Patent Application Publication
(10) Pub. No.: US 2020/0277387 A1
(43) Pub. Date: Sep. 3, 2020

(54) METHODS AND COMPOSITIONS FOR TREATING CANCER

(71) Applicant: RAINIER THERAPEUTICS, INC., San Leandro, CA (US)

(72) Inventor: Graeme CURRIE, San Leandro, CA (US)

(73) Assignee: RAINIER THERAPEUTICS, INC., San Leandro, CA (US)

(21) Appl. No.: 16/805,508

(22) Filed: Feb. 28, 2020

Related U.S. Application Data
(60) Provisional application No. 62/812,929, filed on Mar. 1, 2019.
(60) Provisional application No. 62/856,216, filed on Jun. 3, 2019.
(60) Provisional application No. 62/907,504, filed on Sep. 27, 2019.

(51) Int. Cl. C07K 16/28; A61P 35/00

(52) U.S. Cl. CPC C07K 16/2863; A61P 35/00; A61K 2039/505; C07K 2317/76; C07K 2317/565

(57) ABSTRACT

The present application provides compositions, methods, and kits for treating cancer, including bladder cancer such as luminal bladder cancer, using an FGFR3 inhibitor in combination with a checkpoint inhibitor. In some embodiments, the cancer expresses wild-type FGFR3. The FGFR3 inhibitor may be an antagonistic FGFR3 inhibitor, such as an antagonistic FGFR3 antibody. The checkpoint inhibitor may be a PD1 inhibitor, including a PD1 or PD1 ligand (PD-L1) antibody such as an antagonistic PD1 or PD-L1 antibody.

### PRIORITY CLAIM

This application claims the benefit of U.S. Provisional Patent Application No. 62/812,929, filed Mar. 1, 2019, U.S. Provisional Patent Application No. 62/856,216, filed Jun. 3, 2019, and U.S. Provisional Patent Application No. 62/907,504, filed Sep. 27, 2019, the disclosures of which are incorporated herein by reference in their entirety, including drawings.

### SEQUENCE LISTING

This disclosure includes a sequence listing, which is submitted in ASCII format via EFS-Web, and is hereby incorporated by reference in its entirety. The ASCII copy, created on Feb. 27, 2020, is named SequenceListing.txt and is 10 kilobytes in size.

### BACKGROUND

Urothelial cell carcinoma (UCC)—also known as transitional cell carcinoma (TCC)—occurs in the urinary system (i.e., kidneys, urinary bladder, and accessory organs) and is the most common type of bladder cancer, accounting for 90% of all bladder tumors (Eble 2004). It is the fifth most common cancer in the United States (US) (Costantini 2011) and fourth most common cancer in Europe (Jemal 2011); with an estimated 74,690 new cases and 15,580 deaths occurring in the US in 2014 (American Cancer Society 2018), and an estimated 136,000 new cases and 49,000 deaths occurring in Europe in 2009 (Bellmunt 2009). Clinical and pathological studies have identified two variants of urothelial cell carcinoma of the bladder (UCB) which arise via distinct mechanisms, a low-grade papillary variant and an invasive tumor variant (Wu 2005; Vallot 2010). The low-grade papillary variant accounts for 80% of all UCBs and arises from urothelial hyperplasia. The five-year survival for this tumor type, when treated with surgery and intravesical immunotherapy, is greater than 90% (American Cancer Society 2018). The invasive tumor variant represents 20% of UCBs and has a poor prognosis. Cisplatin-based chemotherapy with either a dose dense M-VAC (Sternberg 2001) or gemcitabine cisplatin (von der Maase 2000) remains the standard treatment for invasive UCC. Despite initial response rates on the order of 50 to 70%, this cancer typically progresses rapidly with a median survival of around 13 to 15 months (von der Maase 2000; Siefker-Radtke 2002). New therapies for UCC are needed.

### SUMMARY

Provided herein are certain embodiments and methods of treating cancer, including for example bladder cancer such as metastatic urothelial cancer (mUC) and upper tract urothelial cancer, in a subject in need thereof comprising administering a therapeutically effective amount of an FGFR3 inhibitor and a therapeutically effective amount of a checkpoint inhibitor such as a PD1 inhibitor. In certain embodiments, the FGFR3 inhibitor binds FGFR3. In other embodiments, the FGFR3 inhibitor binds a ligand for FGFR3, e.g., FGF1, FGF2, or FGF9. In certain embodiments, the FGFR3 inhibitor is an antagonistic FGFR3 antibody, and in certain of these embodiments the antagonistic FGFR3 antibody comprises one or more of a CDR-H1 comprising SEQ ID NO:1, a CDR-H2 comprising SEQ ID NO:2, a CDR-H3 comprising SEQ ID NO:3, a heavy chain variable region comprising SEQ ID NO:7, a heavy chain comprising SEQ ID NO:9, a CDR-L1 comprising SEQ ID NO:4, a CDR-L2 comprising SEQ ID NO:5, a CDR-L3 comprising SEQ ID NO:6, a light chain variable region comprising SEQ ID NO:8, and a light chain comprising the amino acid sequence set forth in SEQ ID NO:10. In certain of these embodiments, the FGFR3 antagonistic antibody is vofatamab. In other embodiments, the antagonistic FGFR3 antibody is selected from the group consisting of PRO-001 and IMC-D11. In certain embodiments, the FGFR3 inhibitor is a small molecule pan-FGFR inhibitor, and in certain of these embodiments the pan-FGFR inhibitor is selected from the group consisting of infigratinib, AZD4547, LY2874455, Pemigatinib, BGJ398, Rogaratinib, PRN1371, Debio 1347, ARQ 087, and JNJ-42756493. In certain embodiments, the PD1 inhibitor binds PD1. In other embodiments, the PD1 inhibitor binds a ligand for PD1, e.g., PDL1 or PDL2. In certain embodiments, the PD1 inhibitor is an antagonistic PD1 antibody, and in certain of these embodiments the antagonistic PD1 antibody is selected from the group consisting of nivolumab, pembrolizumab, CT-011, MEDI-0680, and RMP1-14. In other embodiments, the PD1 inhibitor is an antagonistic PD1 ligand antibody, and in certain of these embodiments the antagonistic PD1 ligand antibody is selected from the group consisting of MEDI-4736, RG7446, BMS-936559, MSB0010718C, and MPDL3280A.

Provided herein in certain embodiments are compositions comprising an FGFR3 inhibitor and a checkpoint inhibitor such as a PD1 inhibitor. In certain of these embodiments, the compositions are pharmaceutical compositions, and in certain embodiments the compositions comprise one or more pharmaceutically acceptable carriers. In certain embodiments, the FGFR3 inhibitor binds FGFR3. In other embodiments, the FGFR3 inhibitor binds a ligand for FGFR3, e.g., FGF1, FGF2, or FGF9. In certain embodiments, the FGFR3 inhibitor is an antagonistic FGFR3 antibody, and in certain of these embodiments the antagonistic FGFR3 antibody comprises one or more of a CDR-H1 comprising SEQ ID NO:1, a CDR-H2 comprising SEQ ID NO:2, a CDR-H3 comprising SEQ ID NO:3, a heavy chain variable region comprising SEQ ID NO:7, a heavy chain comprising SEQ ID NO:9, a CDR-L1 comprising SEQ ID NO:4, a CDR-L2 comprising SEQ ID NO:5, a CDR-L3 comprising SEQ ID NO:6, a light chain variable region comprising SEQ ID NO:8, and a light chain comprising the amino acid sequence set forth in SEQ ID NO:10. In certain of these embodiments, the FGFR3 antagonistic antibody is vofatamab. In other embodiments, the antagonistic FGFR3 antibody is selected from the group consisting of PRO-001 and IMC-D11. In certain embodiments, the FGFR3 inhibitor is a small molecule pan-FGFR inhibitor, and in certain of these embodiments the pan-FGFR inhibitor is selected from the group consisting of infigratinib, AZD4547, LY2874455, Pemigatinib, BGJ398, Rogaratinib, PRN1371, Debio 1347, ARQ 087, and JNJ-42756493. In certain embodiments, the PD1 inhibitor binds PD1. In other embodiments, the PD1 inhibitor binds a ligand for PD1, e.g., PDL1 or PDL2. In certain embodiments, the PD1 inhibitor is an antagonistic PD1 antibody, and in certain of these embodiments the antagonistic PD1 antibody is selected from the group consisting of nivolumab, pembrolizumab, CT-011, MEDI-0680, and RMP1-14. In other embodiments, the PD1 inhibitor is an antagonistic PD1 ligand antibody, and in certain of these embodiments the antagonistic PD1 ligand antibody is selected from the group consisting of MEDI-4736, RG7446, BMS-936559, MSB0010718C, and MPDL3280A.

Provided herein in certain embodiments are methods for treating a cancer expressing wild-type FGFR3 in a subject in need thereof comprising administering to the subject a therapeutically effective amount of an FGFR3 inhibitor in combination with a therapeutically effective amount of a checkpoint inhibitor. In certain embodiments, the FGFR3 inhibitor binds FGFR3. In other embodiments, the FGFR3 inhibitor binds a ligand for FGFR3, e.g., FGF1, FGF2, or FGF9. In certain embodiments, the FGFR3 inhibitor is an antagonistic FGFR3 antibody, and in certain of these embodiments the antagonistic FGFR3 antibody comprises one or more of a CDR-H1 comprising SEQ ID NO:1, a CDR-H2 comprising SEQ ID NO:2, a CDR-H3 comprising SEQ ID NO:3, a heavy chain variable region comprising SEQ ID NO:7, a heavy chain comprising SEQ ID NO:9, a CDR-L1 comprising SEQ ID NO:4, a CDR-L2 comprising SEQ ID NO:5, a CDR-L3 comprising SEQ ID NO:6, a light chain variable region comprising SEQ ID NO:8, and a light chain comprising the amino acid sequence set forth in SEQ ID NO:10. In certain of these embodiments, the FGFR3 antagonistic antibody is vofatamab. In other embodiments, the antagonistic FGFR3 antibody is selected from the group consisting of PRO-001 and IMC-D11. In certain embodiments, the FGFR3 inhibitor is a small molecule pan-FGFR inhibitor, and in certain of these embodiments the pan-FGFR inhibitor is selected from the group consisting of infigratinib, AZD4547, LY2874455, Pemigatinib, BGJ398, Rogaratinib, PRN1371, Debio 1347, ARQ 087, and JNJ-42756493. In certain embodiments, the checkpoint inhibitor is a PD1 inhibitor. In certain of these embodiments, the PD1 inhibitor binds PD1. In other embodiments, the PD1 inhibitor binds a ligand for PD1, e.g., PDL1 or PDL2. In certain embodiments, the PD1 inhibitor is an antagonistic PD1 antibody, and in certain of these embodiments the antagonistic PD1 antibody is selected from the group consisting of nivolumab, pembrolizumab, CT-011, MEDI-0680, and RMP1-14. In other embodiments, the PD1 inhibitor is an antagonistic PD1 ligand antibody, and in certain of these embodiments the antagonistic PD1 ligand antibody is selected from the group consisting of MEDI-4736, RG7446, BMS-936559, MSB0010718C, and MPDL3280A

Provided herein in certain embodiments are methods for treating a subject having cancer expressing wild-type FGFR3 in need thereof comprising (a) screening the subject for a gene signature that correlates with one or more cancer-associated fibroblasts or for p53 expression, (b) determining if the subject has the gene signature that correlates with the one or more cancer-associated fibroblasts or has p53 expression, (c) based on the determining of step (b) and (i) if the subject does not have the gene signature or p53 expression, administering a therapeutically effective amount of an FGFR3 inhibitor in combination with a therapeutically effective amount of a checkpoint inhibitor, and (ii) if the subject does have the gene signature or p53 expression, administering a therapeutically effective amount of an FGFR3 inhibitor in combination with a therapeutically effective amount of a checkpoint inhibitor and an additional anti-cancer agent. In certain embodiments, the FGFR3 inhibitor is an antagonistic FGFR3 antibody. In certain embodiments, the antagonistic FGFR3 antibody comprises CDR-H1 comprising the amino acid sequence set forth in SEQ ID NO:1, CDR-H2 comprising the amino acid sequence set forth in SEQ ID NO:2, CDR-H3 comprising the amino acid sequence set forth in SEQ ID NO:3, and a heavy chain variable region comprising the amino acid sequence set forth in SEQ ID NO:7. In certain embodiments, the antagonistic FGFR3 antibody comprises CDR-L1 comprising the amino acid sequence set forth in SEQ ID NO:4, CDR-L2 comprising the amino acid sequence set forth in SEQ ID NO:5, CDR-L3 comprising the amino acid sequence set forth in SEQ ID NO:6, and a light chain variable region comprising the amino acid sequence set forth in SEQ ID NO:8. In certain embodiments, the FGFR3 inhibitor is vofatamab. In certain embodiments, the checkpoint inhibitor is a PD1 inhibitor. In certain embodiments, the PD1 inhibitor is an antagonistic PD-L1 antibody selected from the group consisting of MEDI-4736, RG7446, BMS-936559, MSB0010718C, and MPDL3280A. In certain embodiments, the PD1 inhibitor is pembrolizumab. In certain embodiments, the cancer is luminal bladder cancer. In certain embodiments, the gene signature includes at least the following genes: FGFR3, TP63, IRS1, SEMA4B, PTPN13, and TMPRS S4. In certain embodiments, the gene signature includes at least the following genes: KRTS, KRT6A, KRT6B, KRT14, UPK3A, UPK3B, FOXA1, and PPARG. In certain embodiments, the gene signature includes at least the following genes: ACTC1, ACTG2, NCC1, DES, FLNC, MFAP4, MYH11, and PCP4.

Provided herein in certain embodiments are kits comprising an FGFR3 inhibitor and a checkpoint inhibitor such as a PD1 inhibitor for use in treating bladder cancer. In certain of these embodiments, the kits further comprise instructions for use.

Provided herein in certain embodiments is the use of an FGFR3 inhibitor and a checkpoint inhibitor such as a PD1 inhibitor for use in formulating a medicament for the treatment of bladder cancer. In certain of these embodiments, the FGFR3 inhibitor and PD1 inhibitor are formulated into a single medicament. In other embodiments, the FGFR3 inhibitor and PD1 inhibitor are formulated into separate medicaments which are administered in combination with one another.

### BRIEF DESCRIPTION OF THE DRAWINGS

This application contains at least one drawing executed in color. Copies of this application with color drawings will be provided by the Office upon request and payment of the necessary fees.

FIG. 1 depicts results from whole transcriptome RNAseq performed on 22 matched biopsies of tumors illustrating two prognostic clusters pre- and post-vofatamab dosing. Cluster 1 was mostly luminal in type and included the majority of respondents. Cluster 2 was more basal in nature and showed clinical benefit consistent with the expected response rate.

FIG. 2 depicts results from whole transcriptome RNAseq performed on 22 matched biopsies of tumors. Gene signatures used to identify basal and luminal subtypes were used to assign 22 paired tumors to a molecular subtype.

FIG. 3 shows inflammatory and immune pathway alterations in responders after vofatamab treatment.

FIG. 4 is a schematic of a phase 1b/phase 2 study design examining the effects of vofatamab plus pembrolizumab in second line mUC.

FIG. 5 shows responsiveness for RECIST 1.1 evaluable patients in the phase 2 study based on WT versus Mut/Fus.

FIG. 6 shows immune changes in responders.

FIG. 7 is a spider plot depicting change in summary of diameter (SoD) of lesions from baseline following vofatamab (B-701) treatment. This plot shows that vofatamab appears to induce an initial apparent increase in tumor size, followed by a sharp reduction in tumor volume.

FIG. 8 illustrates emerging definitions of bladder cancer molecular subtypes.

FIG. 9 shows responsiveness for RECIST 1.1 evaluable patients in the phase 2 study based on molecular subgroup.

FIG. 10 shows association between molecular subtypes and responses to combination therapy.

FIG. 11 shows duration of treatment in the phase 2 study by molecular subgroup.

FIG. 12 shows results of combination vofatamab and pembrolizumab treatment in a male mUC patient with luminal subtype.

FIG. 13 shows progression-free and overall survival for WT FGFR3 patients versus Mut/Fus patients.

FIG. 14 shows survival following combination vofatamab and pembrolizumab treatment. Patients with p53-like tumors (n=5) exhibited poor survival.

### DETAILED DESCRIPTION

The following description of the invention is merely intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein.

There are four single-pass transmembrane tyrosine kinase fibroblast growth factor receptors (FGFR1-4) in humans (Brooks 2012). FGFRs are overexpressed in many cancer types, often due to mutations that confer constitutive activation, making them an attractive target for therapeutic intervention. For example, the FGFR2b antibody FPA144 (FivePrime) is currently under development for the treatment of solid tumors, particularly gastric cancer. Other FGFR2 monoclonal antibodies in early development for cancer treatment include GP369 (Aveo) and HuGAL-FR21 (Galaxy) (Zhao 2010; Bai 2010). A humanized anti-FGFR4 has also been reported to inhibit tumor growth (Bumbaca 2011).

FGFR3 harbors both oncogenic and tumor suppressive properties. FGFR3 is frequently mutated in certain cancers, but in some normal tissues it can limit cell growth and promote cell differentiation (Lafitte 2013). The human FGFR3 antagonistic monoclonal antibody vofatamab (sometimes referred to as B-701 or BM2), was the first FGFR3 targeted antibody to enter clinical development. The variable portion of vofatamab was originally identified through phage display, then recombined with a human IgG1 backbone. Vofatamab binds with high affinity to both wild-type and mutant FGFR3, including the most prevalent mutations found in bladder cancer and achondroplasia (specifically, FGFR3_IIIb^{R248c}, FGFR3-IIIb^{K652E}, FGFR3-III^{Y375C}, FGFR3-IIIb^{S249C}, and FGFR3-IIIb^{G372C}) and gene fusions including FGFR3 TACC3 and FGFR3 BAIAP2L1, while exhibiting no cross-reactivity with other FGFRs. Vofatamab was previously evaluated for safety in patients with t(4:14) translocated multiple myeloma (Clinical Trial NCT01122875). Other FGFR3 inhibitor antibodies that are or have been in clinical or preclinical development include PRO-001 (Prochon), IMC-D11 (ImClone), and ADC LY3076226 (Eli Lilly). Additional FGFR3 antibodies for use in treating cancer and other diseases have been disclosed in, for example, U.S. Pat. No. 8,187,601 (Aveo) and U.S. Pat. No. 7,498,416 (Fibron).

Programmed cell death protein 1 (PD1) is an immune checkpoint receptor from the CD28 superfamily that limits T cell effector functions within tissues following activation by either of its two ligands, PDL1 or PDL2 (Pardo11 2012). PD1 downregulates the immune system by promoting apoptosis in antigen-specific T cells while reducing apoptosis in regulatory (i.e., suppressor) T cells. Certain tumor cells block anti-tumor immune responses in the tumor microenvironment by upregulating ligands for PD1. Blocking the PD1 pathway activates the immune system to attack tumors, and has been shown to induce sustained tumor regression in various tumor types. Accordingly, several PD1 antagonist antibodies are currently approved or in various stages of clinical development. For example, the fully human IgG4 monoclonal PD1 antibody nivolumab (Opdivo®, Bristol-Myers Squibb and Ono Pharmaceutical; also known as ONO-4538, BMS-936558, MDX-1106) is approved for the treatment of unreselectable or metastatic melanoma in patients who no longer respond to other drugs. Nivolumab is also being evaluated for treatment of non-small cell lung cancer (NSCLC) in combination with various chemotherapy regimens. The humanized IgG4 PD1 antibody pembrolizumab (Keytruda®, Merck; also known as MK-3475) is approved for the treatment of several types of cancer, including UCC, NSCLC, and melanoma. Other PD1 antibodies in development include CT-011 (Curetech) and MEDI-0680/AMP-514 (AstraZeneca).

A variety of PD1 ligand (PDL) antibodies are also in development for cancer treatment. For example, the monoclonal IgGlk PDL1 antibody MEDI-4736 (AstraZeneca) is currently in development for the treatment of NSCLC either alone or in combination with the monoclonal CTLA4 antibody tremelimumab (AstraZeneca) or MEDI-0680, the monoclonal IgGlk PDL1 antibody RG7446 (Roche) is in development for use in treating various cancers alone or in combination with Avastin® and Zelboraf®, the fully human monoclonal IgG4 antibody BMS-936559/1VDX-1105 (BMS) is currently in development for the treatment of NSCLC and other cancer types, the fully human IgG1 PDL1 antibody MSB0010718C (Merck Serono) is in development for treating various cancer types, and the Fc-modified monoclonal IgG1 antibody MPDL3280A (Genentech) is currently in development for treatment of NSCLC.

As set forth in the experimental results provided herein, the FGFR3 inhibitor vofatamab has been shown to induce activation of genes associated with a TH1 response and key signaling molecules in immune function. These results suggest that vofatamab activates genes associated with immune cell trafficking pathways. The results also suggest that vofatamab sensitizes patients with mUC with luminal biology to combination treatment with vofatamab and a checkpoint inhibitor, e.g., a PD1 inhibitor. The results provided herein further show that a gene signature associated with cancer-associated fibroblasts appears to be associated with resistance to vofatamab and checkpoint inhibitor combinations, and that patients with p53-like tumors also exhibited resistance to combination treatment. This suggests that patients exhibiting the cancer-associated fibroblast gene signature or with p53-like tumors may be candidates for combination therapies that further include a third agent, e.g., an anti-fibrotic agent.

Provided herein in certain embodiments are methods of treating cancer in a subject in need thereof comprising administering an FGFR3 inhibitor and a PD1 inhibitor. Also provided herein are methods of increasing the effectiveness of a PD1 inhibitor for treating cancer in a subject in need thereof comprising administering an FGFR3 inhibitor, as well as methods of increasing the effectiveness of an FGFR3 inhibitor for treating cancer in a subject in need thereof comprising administering a PD1 inhibitor. An increase in effectiveness of a PD1 or FGFR3 inhibitor may refer to an increase in the therapeutic effect of either inhibitor, a decrease in the required dosage, administration frequency, or administration interval of either inhibitor to obtain a particular level of therapeutic effect, or some combination thereof.

In certain embodiments, the methods provided herein are used to treat a solid cancer, i.e., a cancer that forms a discrete tumor mass. In certain of these embodiments, the cancer being treated is bladder cancer, including for example metastatic bladder cancer (mUC) or upper tract urothelial cancer.

The terms “treat,” “treating,” and “treatment” as used herein with regard to solid cancers may refer to partial or total inhibition of tumor growth, reduction of tumor size, complete or partial tumor eradication, reduction or prevention of malignant growth, partial or total eradication of cancer cells, or some combination thereof. The phrases “patient” and “subject” are used interchangeably herein.

A “subject in need thereof” as used herein refers to a mammalian subject, preferably a human, who has been diagnosed with cancer, is suspected of having cancer, and/or exhibits one or more symptoms associated with cancer. In certain embodiments, the subject may have previously received one or more therapeutic interventions for the treatment of cancer, e.g., chemotherapy.

An “FGFR3 inhibitor” as used herein refers to any molecule that inhibits the activity of FGFR3 either partially or completely. An FGFR3 inhibitor may inhibit FGFR3 specifically, or it may inhibit the activity of other proteins in addition to FGFR3. For example, an FGFR3 inhibitor may also inhibit the activity of other FGFRs. In certain embodiments, an FGFR3 inhibitor may exhibit indirect immunomodulatory activities. For example, an FGFR3 inhibitor may have indirect effects on immune cell trafficking pathways, for example by altering expression and/or activity of TNF alpha or IFN gamma.

An “antagonist antibody” as used herein refers to an antibody that reduces, prevents, or otherwise inhibits interaction between a receptor and its cognate ligand by physically binding to the receptor or the cognate ligand at a binding site and/or an allosteric site on either molecule. In some embodiments, the antagonist antibody interacts at a unique binding site not otherwise involved in the biological regulation of receptor activity by the cognate ligand. An antagonist antibody therefore has affinity for the receptor or the cognate ligand, but no efficacy in promoting the biologic response once bound compared to the cognate ligand binding to the receptor. Binding of the antagonist antibody thereby disrupts the interaction between the receptor and the cognate ligand, and otherwise inhibits the function of an agonist.

In certain embodiments of the methods, compositions, and kits provided herein, the FGFR3 inhibitor inhibits FGFR3 activity by binding to FGFR3. Examples of such FGFR3 inhibitors include, for example, antagonistic FGFR3 antibodies or fusion proteins thereof, inactive forms of the FGFR3 ligand (e.g., truncated or otherwise mutated forms of the FGFR3 ligand) or fusion proteins thereof, small molecules, siRNAs, and aptamers. In certain of these embodiments, the FGFR3 inhibitor specifically binds FGFR3, meaning that the inhibitor exhibits little or no binding to other FGFRs. In other embodiments, the FGFR3 inhibitor binds one or more FGFRs in addition to FGFR3.

In certain preferred embodiments of the methods, compositions, and kits provided herein, the FGFR3 inhibitor is an FGFR3 antagonist antibody, and in certain of these embodiments the FGFR3 antagonist antibody specifically binds FGFR3. The term “antibody” as used herein refers to an immunoglobulin molecule or an immunologically active portion thereof that binds to a specific antigen, for example FGFR3 or PD1. In those embodiments wherein an antibody for use in the present methods, compositions, and kits is a full-length immunoglobulin molecule, the antibody comprises two heavy chains and two light chains, with each heavy and light chain containing three complementary determining regions (CDRs). In those embodiments wherein the antibody is an immunologically active portion of an immunoglobulin molecule, the antibody may be, for example, a Fab, Fab′, Fv, Fab′ F(ab′)2, disulfide-linked Fv, scFv, single domain antibody (dAb), or a diabody. Antibodies for use in the present methods, compositions, and kits may include natural antibodies, synthetic antibodies, monoclonal antibodies, polyclonal antibodies, chimeric antibodies, humanized antibodies, multispecific antibodies, bispecific antibodies, dual-specific antibodies, anti-idiotypic antibodies, or fragments thereof that retain the ability to bind a specific antigen, for example FGFR3 or PD1. Exemplary antibodies include IgA, IgD, IgG1, IgG2, IgG3, IgM and the like. In certain preferred embodiments of the methods, compositions, and kits provided herein, an FGFR3 antibody is an IgG2 antibody.

In certain embodiments, an FGFR3 antagonist antibody for use in the present methods, compositions, and kits comprises a heavy chain variable region comprising one or more complementary determining regions (CDRs) having the sequences set forth in SEQ ID NOs:1-3. In certain of these embodiments, the FGFR3 antagonist antibody comprises all three of these CDR sequences, and in certain of these embodiments the FGFR3 antagonist antibody comprises a heavy chain variable region comprising the amino acid sequence set forth in SEQ ID NO:4. In certain embodiments, the FGFR3 antagonist antibody comprises a light chain variable region comprising one or more CDRs having the sequences set forth in SEQ ID NOs:5-7. In certain of these embodiments, the FGFR3 antagonist antibody comprises all three of these CDR sequences, and in certain of these embodiments the FGFR3 antagonist antibody comprises a light chain variable region comprising the amino acid sequence set forth in SEQ ID NO:8. In certain embodiments, the FGFR3 antagonist antibody comprises all six CDR sequences set forth in SEQ ID NOs:1-3 and 5-7, and in certain of these embodiments the FGFR3 antagonist antibody comprises the heavy chain variable region of SEQ ID NO:4 and the light chain variable region of SEQ ID NO:8. In certain embodiments, the antibody is vofatamab comprising the heavy chain of SEQ ID NO:9 and the light chain of SEQ ID NO:10. In addition to the variable region set forth in SEQ ID NO:7, the heavy chain SEQ ID NO:9 comprises human IgG1. Similarly, the light chain of SEQ ID NO:10 comprises the variable region set forth in SEQ ID NO:8 and human Ig kappa chain C (UniProt P01834).

SEQ ID NO: 1 (H1-CDR): GFTFTSTGIS.
SEQ ID NO: 2 (H2-CDR): GRIYPTSGSTNYADSVKG.
SEQ ID NO: 3 (H3-CDR): ARTYGIYDLYVDYTEYVMDY.
SEQ ID NO: 4 (L1-CDR): RASQDVDTSLA.
SEQ ID NO: 5 (L2-CDR): SASFLYS.
SEQ ID NO: 6 (L3-CDR): QQSTGHPQT.
SEQ ID NO: 7:
EVQLVESGGGLVQPGGSLRLSCAASGFTFTSTGISWVRQAPGKGLEWVGR
IYPTSGSTNYADSVKGRFTISADTSKNTAYLQMNSLRAEDTAVYYCARTY
GIYDLYVDYTEYVMDYWGQGTLV.
SEQ ID NO: 8:
DIQMTQSPSSLSASVGDRVTITCRASQDVDTSLAWYKQKPGKAPKLLIYS
ASFLYSGVPSRFSGSGSGTDFTLTISSLQPEDFATYYCQQSTGHPQTFGQ
GTKVEIKR.
SEQ ID NO: 9:
EVQLVESGGGLVQPGGSLRLSCAASGFTFTSTGISWVRQAPGKGLEWVGR
IYPTSGSTNYADSVKGRFTISADTSKNTAYLQMNSLRAEDTAVYYCARTY
GIYDLYVDYTEYVMDYWGQGTLVTVSSASTKGPSVFPLAPSSKSTSGGTA
ALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKS
LSLSPGK.
SEQ ID NO. 10:
DIQMTQSPSSLSASVGDRVTITCRASQDVDTSLAWYKQKPGKAPKLLIYS
ASFLYSGVPSRFSGSGSGTDFTLTISSLQPEDFATYYCQQSTGHPQTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

In other embodiments, an FGFR3 antagonist antibody for use in the present methods, compositions, and kits may be PRO-001, IMC-D11, or an FGFR3 antagonistic antibody as disclosed in U.S. Pat. No. 8,187,601 (Aveo) or U.S. Pat. No. 7,498,416 (Fibron). In certain embodiments, an FGFR3 antagonist antibody for use in the present methods, compositions, and kits may be lyophilized.

In certain embodiments of the methods, compositions, and kits provided herein, the FGFR3 inhibitor inhibits FGFR3 activity by binding to a ligand for FGFR3, e.g., FGF1, FGF2, or FGF9. Examples of such FGFR3 inhibitors include, for example, antibodies that specifically bind an FGFR3 ligand or fusion proteins thereof, soluble forms of FGFR3 comprising all or part of the FGFR3 extracellular domain or fusion proteins thereof, truncated forms of FGFR3 lacking all or part of the intracellular domains required for downstream signaling or fusion proteins thereof, small molecules, siRNAs, and aptamers.

In certain embodiments of the methods, compositions, and kits provided herein, the FGFR3 inhibitor is a pan-FGFR inhibitor, meaning that it binds to and inhibits the activity of one or more FGFRs in addition to FGFR3. In certain of these embodiments, the FGFR3 inhibitor may be a small molecule pan-FGFR inhibitor selected from the group consisting of infigratinib (BGJ398, Novartis), AZD4547 (AstraZeneca), LY2874455 (Eli Lilly), Debio 1347 (Debiopharm), ARQ 087 (ArQule), JNJ-42756493 (Janssen), and PRN1371 (Principia).

In certain embodiments of the methods, compositions, and kits provided herein, the FGFR3 inhibitor inhibits FGFR3 activity by blocking downstream tyrosine kinase activity. For example, a non-selective tyrosine kinase inhibitor such as dovitinib, lucitinib, ponatinib, nintedanib, ponatinib, or ENMD-2076 may be utilized as an FGFR3 inhibitor.

A “PD1 inhibitor” as used herein refers to any molecule that inhibits the activity of PD1 either partially or completely. A PD1 inhibitor may inhibit PD1 specifically, or it may inhibit the activity of other proteins in addition to PD1. For example, a PD1 inhibitor may also inhibit the activity of other immune checkpoint molecules.

In certain embodiments of the methods, compositions, and kits provided herein, the PD1 inhibitor inhibits PD1 activity by binding to PD1. Examples of such PD1 inhibitors include, for example, antagonistic PD1 antibodies or fusion proteins thereof, inactive forms of a PD1 ligand (e.g., truncated or otherwise mutated forms of PDL1 or PDL2) or fusion proteins thereof (e.g., AMP-224 (GlaxoSmithKline, Amplimmune)), small molecules, siRNAs, and aptamers.

In certain embodiments of the methods, compositions, and kits provided herein, the PD1 inhibitor is a PD1 antibody, and in certain of these embodiments the PD1 antagonist antibody specifically binds PD1. In certain embodiments, the PD1 antagonistic antibody is selected from the group consisting of nivolumab, pembrolizumab, CT-011, MEDI-0680, and RMP1-14.

In certain embodiments of the methods, compositions, and kits provided herein, the PD1 inhibitor inhibits PD1 activity by binding to one or more ligands for PD1, i.e., PDL1 or PDL2. Examples of such PD1 inhibitors include, for example, PD1 ligand antibodies or fusion proteins thereof, soluble forms of PD1 comprising all or part of the PD1 extracellular domain or fusion proteins thereof, truncated forms of PD1 lacking all or part of the intracellular domains required for downstream signaling or fusion proteins thereof, small molecules, siRNAs, and aptamers.

In certain embodiments of the methods, compositions, and kits provided herein, the PD1 inhibitor is a PD1 ligand antibody, and in certain of these embodiments the PD1 ligand antibody specifically binds the PD1 ligand. In certain embodiments, the PD1 ligand antibody is selected from the group consisting of MEDI-4736, RG7446, BMS-936559, MSB0010718C, and MPDL3280A.

In certain embodiments of the methods provided herein, the FGFR3 inhibitor and PD1 inhibitor are administered together as part of the same composition. In other embodiments, the FGFR3 inhibitor and PD1 inhibitor are administered separately, i.e., as separate compositions. In these embodiments, the inhibitors may be administered simultaneously or sequentially, and may be administered via the same or different routes. In those embodiments where the inhibitors are administered sequentially, they may be administered at the same or different intervals. For example, one inhibitor may be administered more frequently than the other, or may be administered over a longer time course. In certain of these embodiments, one inhibitor may be administered one or more times prior to the first administration of the second inhibitor. When administration of the second inhibitor is initiated, administration of the first inhibitor may either cease or continue for all or part of the course of administration of the second inhibitor. In certain embodiments wherein the FGFR3 inhibitor is an FGFR3 antagonist antibody, the antibody may be administered two or more times per day, daily, two or more times per week, weekly, biweekly (i.e., every other week), every third week, or monthly. In certain embodiments, the antibody is administered weekly, bi-weekly, or every third week. In certain embodiments wherein the PD1 inhibitor is a PD1 antagonist antibody, the antibody may be administered two or more times per day, daily, two or more times per week, weekly, bi-weekly, every third week, or monthly. In certain embodiments, the PD1 inhibitor is administered bi-weekly. In certain embodiments, the FGFR3 inhibitor and/or the PD1 inhibitor may be administered for a specific time course determined in advance. For example, the FGFR3 and/or PD1 inhibitors may be administered for a time course of 1 day, 2 days, 1 week, 2 weeks, 4 weeks, or 8 weeks. In other embodiments, the FGFR3 and/or PD1 inhibitors may be administered indefinitely, or until a specific therapeutic benchmark is reached. For example, the FGFR3 and/or PD1 inhibitors may be administered until tumor growth is arrested or reversed, until one or more tumors are eliminated, or until the number of cancer cells are reduced to a specific level.

A “therapeutically effective amount” of a composition as used herein is an amount of a composition that produces a desired therapeutic effect in a subject, such as treating cancer. In certain embodiments, the therapeutically effective amount is an amount of the composition that yields maximum therapeutic effect. In other embodiments, the therapeutically effective amount yields a therapeutic effect that is less than the maximum therapeutic effect. For example, a therapeutically effective amount may be an amount that produces a therapeutic effect while avoiding one or more side effects associated with a dosage that yields maximum therapeutic effect. A therapeutically effective amount for a particular composition will vary based on a variety of factors, including but not limited to the characteristics of the therapeutic composition (e.g., activity, pharmacokinetics, pharmacodynamics, and bioavailability), the physiological condition of the subject (e.g., age, body weight, sex, disease type and stage, medical history, general physical condition, responsiveness to a given dosage, and other present medications), the nature of any pharmaceutically acceptable carriers in the composition, and the route of administration. One skilled in the clinical and pharmacological arts will be able to determine a therapeutically effective amount through routine experimentation, namely by monitoring a subject's response to administration of a composition and adjusting the dosage accordingly. For additional guidance, see, e.g., Remington: The Science and Practice of Pharmacy, 22nd Edition, Pharmaceutical Press, London, 2012, and Goodman & Gilman's The Pharmacological Basis of Therapeutics, 12th Edition, McGraw-Hill, New York, N.Y., 2011, the entire disclosures of which are incorporated by reference herein.

In certain embodiments of the methods provided herein, a therapeutically effective amount of an FGFR3 inhibitor or a PD1 inhibitor may be a dosage at which the molecule is capable of generating a therapeutic response (e.g., reducing or eliminating tumor growth) as a monotherapy, i.e., when administered alone. In certain of these embodiments, the therapeutically effective amount may be a dosage that has previously been determined to be optimal or near optimal for cancer treatment. For example, where the FGFR3 inhibitor is vofatamab, the antibody may be administered at a dosage of about 10 to 50 mg/kg every two to four weeks, and in certain of these embodiments the antibody may be administered at a dosage of about 20 to 40 mg/kg every two to four weeks, or about 30 mg/kg every three weeks. In other embodiments, a therapeutically effective amount of an FGFR3 inhibitor or a PD1 inhibitor may be lower than the dosage at which the molecule would normally be administered for use as a monotherapy, i.e., a suboptimal dose. In certain of these embodiments, administration of the suboptimal dosage of FGFR3 or PD1 inhibitor may result in decreased side effects versus the standard dosage when administered alone. For example, administration of suboptimal dosage of FGFR3 or PD1 inhibitors may result in decreased occurrence or severity of pruritus, colitis, or pneumonia versus administration of the optimal dosage of either inhibitor alone. In certain embodiments, one of an FGFR3 inhibitor and a PD1 inhibitor may be administered at a dosage that has been determined to be optimal for cancer treatment when administered alone, while the other is administered at a dosage that is suboptimal for treatment when administered alone. In certain embodiments, the dosage of the FGFR3 inhibitor or PD1 inhibitor may change over the course of the treatment regimen. For example, one or both of the FGFR3 inhibitor and PD1 inhibitor may be administered at higher dosage at the start of treatment (e.g., a loading phase), followed by a lower dosage later in treatment.

An FGFR3 inhibitor, PD1 inhibitor, or composition comprising both an FGFR3 inhibitor and a PD1 inhibitor may be delivered to a subject by any administration pathway known in the art, including but not limited to parenteral, oral, aerosol, enteral, nasal, ophthalmic, parenteral, or transdermal (e.g., topical cream or ointment, patch). “Parenteral” refers to a route of administration that is generally associated with injection, including intravenous, intraperitoneal, subcutaneous, infraorbital, infusion, intraarterial, intracapsular, intracardiac, intradermal, intramuscular, intrapulmonary, intraspinal, intrasternal, intrathecal, intrauterine, subarachnoid, subcapsular, transmucosal, or transtracheal. In certain embodiments wherein the FGFR3 inhibitor is an FGFR3 antagonist antibody, including for example vofatamab, the FGFR3 inhibitor is administered intravenously. In certain embodiments wherein the PD1 inhibitor is a PD1 antagonist antibody, the PD1 inhibitor is administered intraperitoneally.

In certain embodiments, FGFR3 inhibitors, PD1 inhibitors, or compositions comprising both FGFR3 and PD1 inhibitors may be formed into oral dosage units, such as for example tablets, pills, or capsules. In certain embodiments, FGFR3 inhibitor, PD1 inhibitor, or FGFR3 and PD1 inhibitor compositions may be administered via a time release delivery vehicle, such as, for example, a time release capsule. A “time release vehicle” as used herein refers to any delivery vehicle that releases active agent over a period of time rather than immediately upon administration. In other embodiments, FGFR3 inhibitor, PD1 inhibitor, or FGFR3 and PD1 inhibitor compositions may be administered via an immediate release delivery vehicle.

In certain embodiments of the methods provided herein, subjects receiving FGFR3 inhibitor and PD1 inhibitor may receive additional therapies, including for example chemotherapy or immunotherapy, or a third agent such as an anti-fibrotic agent, before, during, or after treatment with FGFR3 and PD1 inhibitors. In those embodiments where the subject receives additional therapies during treatment with FGFR3 and PD1 inhibitors, the additional therapies may be administered simultaneously or sequentially with the FGFR3 inhibitor and/or PD1 inhibitor.

Provided herein in certain embodiments are compositions comprising a therapeutically effective amount of an FGFR3 inhibitor and a therapeutically effective amount of a PD1 inhibitor. In certain embodiments, these compositions further comprise one or more pharmaceutically acceptable carriers, or are formulated for administration with one or more pharmaceutically acceptable carriers. Also provided herein are kits comprising an FGFR3 inhibitor and a PD1 inhibitor for use in carrying out the methods disclosed herein, e.g., for treating cancer.

In certain embodiments of the compositions and kits provided herein, an FGFR3 inhibitor or PD1 inhibitor may be present in the composition or kit at a dosage at which it is capable of generating a therapeutic response (e.g., reducing or eliminating tumor growth) when administered alone. In certain of these embodiments, the FGFR3 or PD1 inhibitor may be present at a dosage that has previously been determined to be optimal or near optimal for cancer treatment. For example, where the FGFR3 inhibitor is vofatamab, the composition or kit may be formulated to deliver a dosage of about 10 to 50 mg/kg of vofatamab to the subject, and in certain of these embodiments the composition or kit may be formulated to deliver a dosage of about 20 to 40 mg/kg or about 30 mg/kg of vofatamab to the subject. In other embodiments, the FGFR3 or PD1 inhibitor may be present at a dosage that is lower than that at which it would normally be present in a composition or kit for cancer treatment (i.e., a suboptimal dose).

A “pharmaceutically acceptable carrier” as used herein refers to a pharmaceutically acceptable material, composition, or vehicle that is involved in carrying or transporting a compound or molecule of interest from one tissue, organ, or portion of the body to another tissue, organ, or portion of the body. A pharmaceutically acceptable carrier may comprise a variety of components, including but not limited to a liquid or solid filler, diluent, excipient, solvent, buffer, encapsulating material, surfactant, stabilizing agent, binder, or pigment, or some combination thereof. Each component of the carrier must be “pharmaceutically acceptable” in that it must be compatible with the other ingredients of the composition and must be suitable for contact with any tissue, organ, or portion of the body that it may encounter, meaning that it must not carry a risk of toxicity, irritation, allergic response, immunogenicity, or any other complication that excessively outweighs its therapeutic benefits.

Examples of pharmaceutically acceptable carriers that may be used in conjunction with the compositions provided herein include, but are not limited to, (1) sugars, such as lactose, glucose, sucrose, or mannitol; (2) starches, such as corn starch and potato starch; (3) cellulose and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols such as propylene glycol; (11) polyols such as glycerin, sorbitol, mannitol and polyethylene glycol; (12) esters, such as ethyl oleate and ethyl aurate; (13) disintegrating agents such as agar or calcium carbonate; (14) buffering or pH adjusting agents such as magnesium hydroxide, aluminum hydroxide, sodium chloride, sodium lactate, calcium chloride, and phosphate buffer solutions; (15) alginic acid; (16) pyrogen-free water; (17) isotonic saline; (18) Ringer's solution; (19) alcohols such as ethyl alcohol and propane alcohol; (20) paraffin; (21) lubricants, such as talc, calcium stearate, magnesium stearate, solid polyethylene glycol, or sodium lauryl sulfate; (22) coloring agents or pigments; (23) glidants such as colloidal silicon dioxide, talc, and starch or tri-basic calcium phosphate; (24) other non-toxic compatible substances employed in pharmaceutical compositions such as acetone; and (25) combinations thereof.

Compositions comprising an FGFR3 inhibitor, a PD1 inhibitor, or a combination of an FGFR3 inhibitor and a PD1 inhibitor may be formulated into a suitable dosage form, including for example solutions or suspensions in an aqueous or non-aqueous liquid, oil-in-water or water-in-oil liquid emulsions, capsules, cachets, pills, tablets, lozenges, powders, granules, elixirs or syrups, or pastilles. In certain embodiments, the compositions may be formulated as time release delivery vehicles, such as, for example, a time release capsule. A “time release vehicle” as used herein refers to any delivery vehicle that releases an active agent over a period of time rather than immediately upon administration. In other embodiments, the compositions may be formulated as immediate release delivery vehicles.

Provided herein in certain embodiments are kits for carrying out the methods disclosed herein. In certain embodiments, the kits provided herein comprise an FGFR3 inhibitor and a PD1 inhibitor. In certain embodiments, the FGFR3 inhibitor and PD1 inhibitor may be present in the kit in a single composition. In other embodiments, the FGFR3 inhibitor and PD1 inhibitor may be present in separate compositions. The kits may comprise additional therapeutic or nontherapeutic compositions. In certain embodiments, the kits comprise instructions in a tangible medium.

Provided herein in certain embodiments are an FGFR3 inhibitor and a PD1 inhibitor for use in the treatment of cancer. Also provided are an FGFR3 inhibitor for use in the treatment of cancer in combination with a PD1 inhibitor, and a PD1 inhibitor for use in the treatment of cancer in combination with an FGFR3 inhibitor. In certain of these embodiments, the cancer is urothelial cancer, such as mUC. In certain of these embodiments, the cancer is luminal bladder cancer. For example, subjects needing treatment for cancer having a certain gene signature may be candidates for treatment with the FGFR3 inhibitor and the PD1 inhibitor of the present disclosure. As another example, subjects needing treatment for cancer having another certain gene signature may not be candidates for treatment with the FGFR3 inhibitor and the PD1 inhibitor of the present disclosure and may rather have treatment which includes the third agent of the present disclosure. In some embodiments, the another certain gene signatures include gene signatures associated with cancer-associated fibroblasts, and p53-like tumors. In certain embodiments, the gene signature is an FGFR3 gene signature and includes at least the following genes: FGFR3, TP63, IRS1, SEMA4B, PTPN13, and TMPRSS4. In certain embodiments, the gene signature is a p53-like gene signature and includes at least the following genes: KRTS, KRT6A, KRT6B, KRT14, UPK3A, UPK3B, FOXA1, and PPARG. In certain embodiments, the gene signature is a cancer-associated fibroblast (CAF) gene signature and includes at least the following genes: ACTC1, ACTG2, NCC1, DES, FLNC, MFAP4, MYH11, and PCP4.

Provided herein in certain embodiments are methods for treating a subject having cancer expressing wild-type FGFR3 in need thereof comprising (a) screening the subject for a gene signature that correlates with one or more cancer-associated fibroblasts or for p53 expression, (b) determining if the subject has the gene signature that correlates with the one or more cancer-associated fibroblasts or has p53 expression, (c) based on the determining of step (b) and (i) if the subject does not have the gene signature or p53 expression, administering a therapeutically effective amount of an FGFR3 inhibitor in combination with a therapeutically effective amount of a checkpoint inhibitor, and (ii) if the subject does have the gene signature or p53 expression, administering a therapeutically effective amount of an FGFR3 inhibitor in combination with a therapeutically effective amount of a checkpoint inhibitor and an additional anti-cancer agent. In certain embodiments, the FGFR3 inhibitor is an antagonistic FGFR3 antibody. In certain embodiments, the antagonistic FGFR3 antibody comprises CDR-H1 comprising the amino acid sequence set forth in SEQ ID NO:1, CDR-H2 comprising the amino acid sequence set forth in SEQ ID NO:2, CDR-H3 comprising the amino acid sequence set forth in SEQ ID NO:3, and a heavy chain variable region comprising the amino acid sequence set forth in SEQ ID NO:7. In certain embodiments, the antagonistic FGFR3 antibody comprises CDR-L1 comprising the amino acid sequence set forth in SEQ ID NO:4, CDR-L2 comprising the amino acid sequence set forth in SEQ ID NO:5, CDR-L3 comprising the amino acid sequence set forth in SEQ ID NO:6, and a light chain variable region comprising the amino acid sequence set forth in SEQ ID NO:8. In certain embodiments, the FGFR3 inhibitor is vofatamab. In certain embodiments, the checkpoint inhibitor is a PD1 inhibitor. In certain embodiments, the PD1 inhibitor is an antagonistic PD-L1 antibody selected from the group consisting of MEDI-4736, RG7446, BMS-936559, MSB0010718C, and MPDL3280A. In certain embodiments, the PD1 inhibitor is pembrolizumab. In certain embodiments, the cancer is luminal bladder cancer.

Provided herein in certain embodiments is the use of an FGFR3 inhibitor and a PD1 inhibitor in the manufacture of a medicament for treating cancer. Also provided are the use of an FGFR3 inhibitor in the manufacture of a medicament for treating cancer in combination with a PD1 inhibitor, and the use of a PD1 inhibitor in the manufacture of a medicament for treating cancer in combination with an FGFR3 inhibitor.

The term “about” as used herein means within 10% of a stated value or range of values.

One of ordinary skill in the art will recognize that the various embodiments described herein can be combined. For example, steps from the various methods of treatment disclosed herein may be combined in order to achieve a satisfactory or improved level of treatment.

From the foregoing, it will be appreciated that specific embodiments of the invention have been described herein for purposes of illustration, but that various modifications may be made without deviating from the scope of the invention. Accordingly, the invention is not limited except as by the appended claims.

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1: Gene Expression Profiling in Wild Type and Mutant FGFR3 Metastatic Urothelial Cancer Treated with Combination Therapy with Vofatamab and Pembrolizumab

Among patients with metastatic urothelial cancer (mUC), FGFR3 mutations (mutFGFR3) are present in 15-20% of bladder cancer patients and around 35% of upper tract cancers. Studies suggest that these tumors may respond better to FGFR inhibition than to immunotherapy.

Patients who failed prior treatment for mUC were treated with vofatamab. Patients were treated with a loading dose (25 mg/kg) of vofatamab with a biopsy pre- and 14 days post-treatment, followed by combination therapy with pembrolizumab to cohorts of patients with and without mutFGFR3 or gene infusions. Core biopsies (2x18G or 3-4x20G) were fixed in 10% neutral buffered formalin and embedded in paraffin. The biopsy core was then isolated from the paraffin block of these formalin fixed paraffin embedded (FFPE) samples.

DNA and RNA were isolated from the samples for whole transcriptome RNAseq and DNA sequencing on the Ion Torrent platform. Whole transcriptome RNAseq was performed on samples from 22 patients with matched pre- and post-treatment biopsies (17 WT, 5 Mut Fusion) using Ion Torrent's AmpliSeqRNA platform (Thermo Fisher, Inc.) and an Ion Proton sequencer (Thermo Fisher, Inc). RNA was then transcribed into cDNA using the SuperScript® VILO™ kit. cDNA was amplified using the Ion Ampliseq Transcriptome Human Gene Expression Core panel, followed by ligation of adapters and barcodes to amplicons and purification. Purified libraries were quantified using the Ion Library Quantification kit (Thermo Fisher, Inc.). Libraries were then diluted to 100 pM, pooled, and amplified on the Ion Sphere™ particles (ISP) using emulsion PCR and enriched on the IonChef (Thermo Fisher, Inc). Template positive ISPs were subsequently loaded into Ion PI chips and run on the Proton instrument. Targeted DNA sequencing of cancer related genes was performed using the AmpliSeq Comprehensive Cancer panel (409 tumor suppressor genes and oncogenes including FGFR3) or the Ion AmpliSeg™ Cancer Hotspot Panel v2.

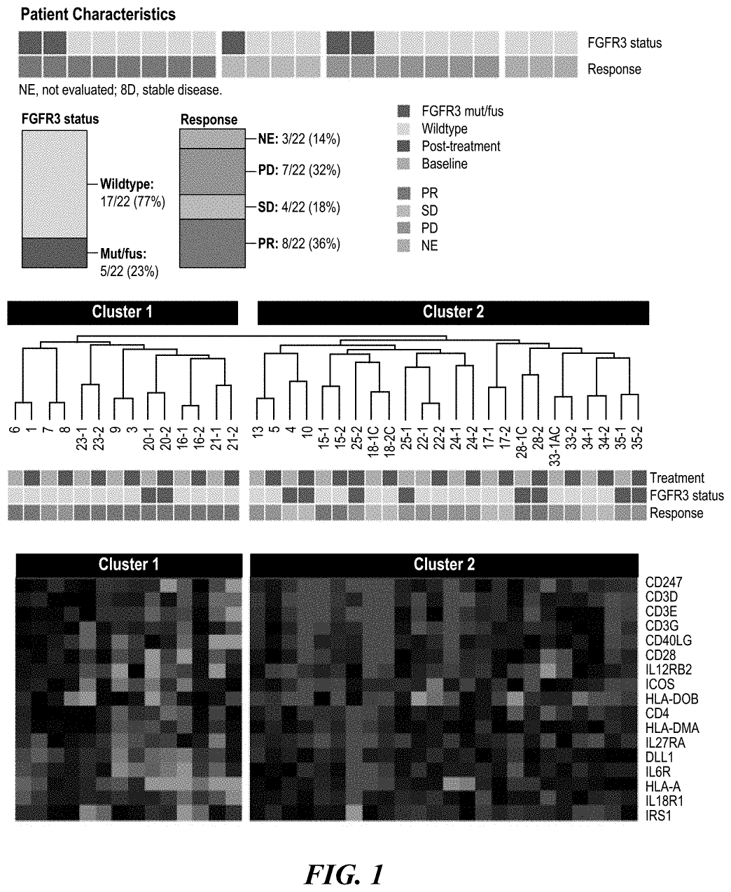

RNA-Seq gene expression analysis was performed using AmpliSeqRNA analysis plugin in the Torrent Suite Software. This plugin aligns raw sequence reads to a human reference genome that contains 20,802 RefSeq transcripts (hg19 Ampliseq Transcriptome_ERCC_V1.fasta) using the Torrent Mapping Alignment Program (TMAP). Then, the number of reads mapped per gene were counted to generate raw counts files and normalized reads per gene per million mapped reads (RPM) files. Tumor subtype assignments were made using the MD Anderson one nearest neighbor (oneNN) classifier. Eight tumors exhibited partial response (PR), four exhibited stable disease (SD), seven exhibited progressed disease (PD), and three were not classified (FIG. 1).

To perform tests for differential expression (DE) between pre- and post-treatment tumors, the Bioconductor package DESeq with Negative Binomial model was used. The Benjamini-Hochberg method was used to control the false discovery rate (FDR). Unsupervised cluster analysis of baseline tissue revealed the presence of two clusters: cluster 1 was enriched for responders (6 PR, 1 SD), compared with cluster 2 (2 PR, 4 SD, 6 PD) (FIG. 1). Significantly differentially expressed genes from cluster 1 and 2 were extracted and analyzed by Ingenuity Pathway Analysis (Sigma).

To conduct pathway analysis, published gene expression signatures characteristic of immune infiltration, inflammatory cell trafficking, IFN pathway activation, TH1 pathway activation, and FGFR3 gene expression signatures were evaluated. Functional and pathway analyses were performed using Ingenuity Pathway Analysis (IPA) software (Ingenuity® Systems, CA), which contains a database for identifying networks and pathways of interest in genomic data. “Transcriptional factors as molecule type in upstream regulator” categories within IPA were used to interpret the biological properties of the bladder tumor subtypes. For upstream regulator analyses, IPA performs statistical analyses for overlap p values and an activation z-score. Based on the IPA knowledge database, p-values and z-scores can be calculated based on how many targets of each transcriptional factor will be overwrapped (p-values) and the extent of concordance of the known effects (activation or inhibition) of the targets in the gene lists (z-score).

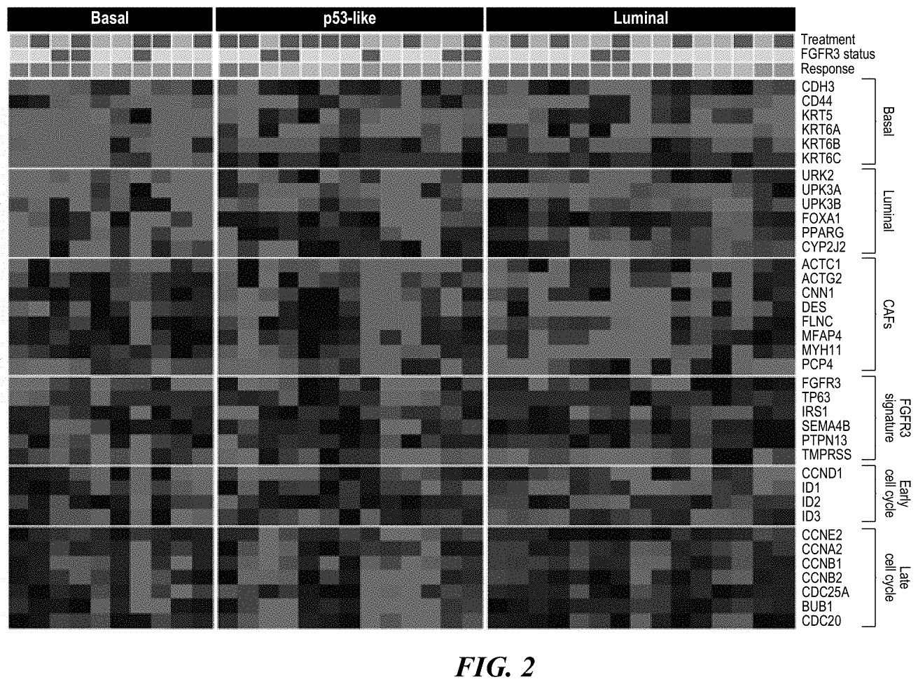

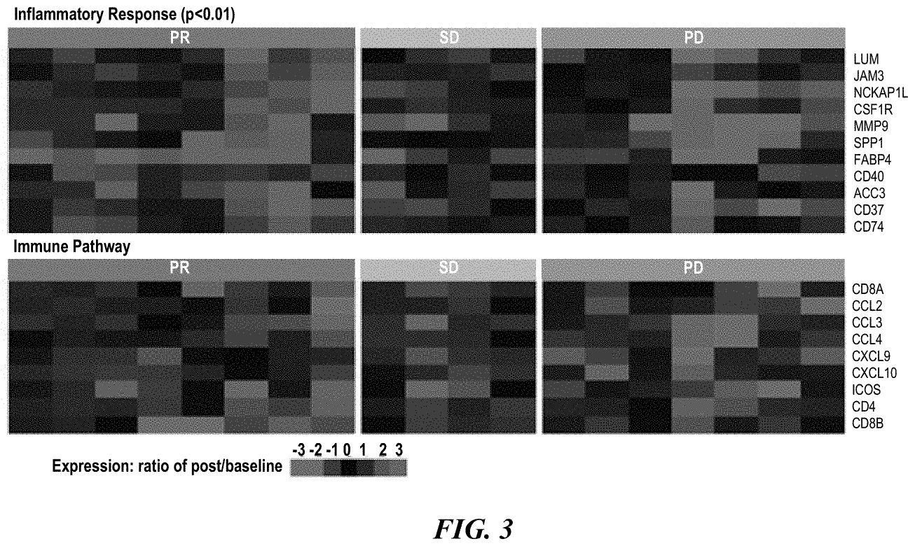

Gene signatures used to identify basal and luminal subtypes were used on baseline tissue to assign 22 paired tumors to molecular subtypes (FIG. 2). Of the responding tumors, six were luminal and two were basal (FIG. 2); see also Denrauer 2014. Differential expression analyses in responders indicated that vofatamab impacts inflammatory and immune pathways and immune cell trafficking (FIG. 3).

Collectively, these data indicate that luminal biology may sensitize subjects to combination treatment with vofatamab and pembrolizumab regardless of the presence or absence of mutFGFR3, suggesting a role for this combination in wtFGFR3 mUC. The effect of the combination on tumors with basal biology also confirmed effectiveness. These biological effects may also be important in earlier stage bladder cancer, e.g., non-muscle-invasive bladder cancer (NMIBC). If vofatamab has effects on immune cell trafficking, it may also show benefit in NMIBC either following or in combination with BCG treatment.

### Example 2: Clinical Evaluation of Vofatamab Monotherapy and Combination Therapy with Pembrolizumab in Subjects with mUC

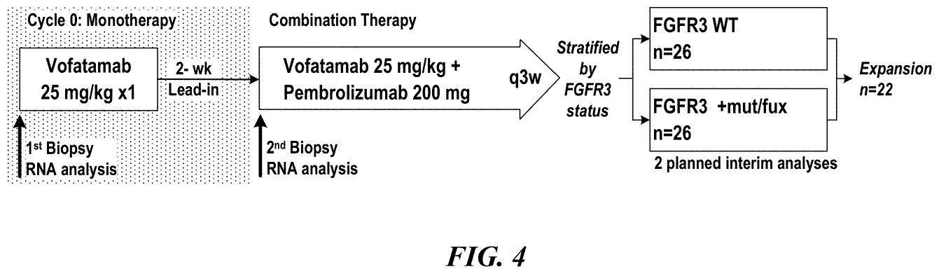

The FIERCE-22 Phase 1b/2 clinical study was designed to evaluate vofatamab in combination with pembrolizumab. FIG. 4 shows a schematic of the study. Subjects received vofatamab monotherapy over a two-week lead-in, with paired pre- and post-biopsies, followed by initiation of combination therapy. After the lead-in period, combination therapy was initiated.

An initial interim analysis was planned after enrollment of 26 patients. 28 patients were enrolled in the study. A second interim analysis is planned after enrollment of 52 patients. To qualify for enrollment, mUC patients needed to be anti-PD-/L1 naïve with measurable disease, ECOG greater than two, and progression on one or more lines of prior platinum-based chemotherapy or recurrence within 12 months or less of (neo)adjuvant chemotherapy. The demographics and treatment history of the initial 28 patients is set forth in Table 1.

TABLE 1
Baseline demographics and treatment history:

| | | |
|---|---|---|
| Total | WT | Mut/Fus |
| (n = 28) | (n = 20) | (n = 8) |

Baseline demographics

| Male (n) (%)) | | 17 | (61%) | 11 | (55%) | 6 | (75% |
|---|---|---|---|---|---|---|---|
| Median age (years) | | 62 | | 60 | | 65 | |
| White (n, %) | | 27 | (96%) | 19 | (95%) | 8 | (100%) |
| Median time from mUC onset | | 8.7 | | 7.2 | | 12.1 | |
| (months) | | | | | | | |
| ECOG score | 0 | 14 | (50%) | 10 | (50%) | 4 | (50%) |
| (n, %) | 1 | 14 | (50%) | 10 | (50%) | 4 | (50%) |
| Hemoglobin | <10 g/dL | 4 | (14%) | 3 | (15%) | 1 | (13%) |
| (n, %) | ≥10 g/dL | 23 | (82%) | 17 | (85%) | 6 | (75%) |
| Liver metastases (n, %) | | 9 | (32%) | 8 | (40%) | 1 | (13%) |
| Bellmunt score | 0 | 9 | (32%) | 6 | (30%) | 3 | (38%) |
| (n, %) | 1 | 12 | (43%) | 8 | (40%) | 4 | (50%) |
| | 2 | 6 | (21%) | 5 | (25%) | 1 | (13%) |
| | 3 | 1 | (4%) | 1 | (5%) | 0 | (0%) |

Treatment history

| | | | | | | | |
|---|---|---|---|---|---|---|---|
| Median # of | | 1 | (1-5) | 1 | (1-5) | 2 | (1-3) |
| prior lines of | | | | | | | |
| therapy (range) | | | | | | | |
| # of prior | 1 | 17 | (61%) | 14 | (70%) | 3 | (38%) |
| regiments | 2 | 7 | (25%) | 3 | (15%) | 4 | (50%) |
| (n, %) | ≥3 | 4 | (14%) | 3 | (15%) | 1 | (13%) |

| | | | | | | |
|---|---|---|---|---|---|---|
| PD as best response to prior | 9 | (32%) | 6 | (30%) | 3 | (38%) |
| therapy (n, %) | | | | | | |

| | | | |
|---|---|---|---|
| Median time from most recent | 3.4 | 4.0 | 2.8 |
| line (months) | | | |

Primary endpoints were safety and efficacy. Overall response rate (ORR) was assessed by investigators using RECIST 1.1, with any biopsied lesion begin considered non-target for evaluation. The disposition of the initial 28 patients is set forth in Table 2. Median treatment exposure was prolonged, with the majority of patients initiating eight or more treatment cycles.

TABLE 2
Disposition - Phase 2:

| | | Total | | WT | | Mut/Fus | |
|---|---|---|---|---|---|---|---|
| | | (n = 28) | | (n = 20) | | (n = 8) | |
| Patients ongoing in study (n, %) | | 14 | (50%) | 10 | (50%) | 4 | (50%) |
| On treatment (n, %) | | 6 | (21%) | 5 | (25%) | 1 | (13%) |
| Drug exposure | Median treatment | 143 | | 147 | | 126 | |
| (study ongoing) | duration (days) | | | | | | |
| | Median | 8 | (2-16) | 8 | (2-16) | 6 | (3-10) |
| | vofatamab doses | | | | | | |
| | (n, range) | | | | | | |
| | Median | 7 | (1-15) | 7 | (1-15) | 5 | (2-9) |
| | pembrolizumab | | | | | | |
| | doses (n, range) | | | | | | |
| Reasons for ending | Death | 11 | (39%) | 8 | (40%) | 3 | (38%) |
| treatment (n, %) | PD per RECIST | 13 | (46%) | 9 | (45%) | 4 | (50%) |
| | 1.1 | | | | | | |
| | Withdrawal of | 2 | (7%) | 1 | (5%) | 1 | (13%) |
| | consent | | | | | | |
| | Investigator | 0 | | 0 | | 0 | |
| | decision | | | | | | |
| | TEAE* | 2 | (7%) | 1 | (5%) | 1 | (13%) |

| | | | | | |
|---|---|---|---|---|---|
| Other | 2 | (7%) | 2 | (10%) | 0 |
| Lost to follow-up | 1 | (4%) | 1 | (4%) | 0 |

| | | | |
|---|---|---|---|
| RECIST 1.1 evaluable (n) | 22 | 15 | 7 |

*Worsening anemia and tracheobronchitis

The clinical response of the initial 28 patients of Phase 2 is set forth in Table 3 and the clinical response for the evaluable population from RECIST 1.1 is set forth in Table 4.

TABLE 3
Clinical Response - Phase 2:

| | Total | | WT | | Mut/Fus | |
|---|---|---|---|---|---|---|
| | (n = 28) | | (n = 20) | | (n = 8) | |
| Median PFS, mo | 5.9 | (3.6, 8.0) | 5.9 | (3.6, 9.7) | 5.0 | (2.3, 8.0) |
| (95% CI) | | | | | | |
| Median Duration | 6.3 | (4.4, NE) | 6.4 | (6.2, NE) | 4.4 | (NE) |
| of Response (DOR), | | | | | | |
| mo (95% CI) | | | | | | |

| | | | |
|---|---|---|---|
| Median OS, mo | NR | NR | NR |
| Proportion alive at | 57.2 | 57.9 | 62.5 |
| 12 mo. % | | | |

TABLE 5
Commonly Occurring TEAEs in ≥15% of Patients in Phase 1b/2:

| | | |
|---|---|---|
| Medical Dictionary for | | |
| Regulatory Activities, | Any Grade, n (%) | Grade ≥3, n (%) |
| Preferred Term | n = 36* | n = 36 |

| Anemia | 15 | (42) | 4 | (11)¶ |
|---|---|---|---|---|
| Fatigue | 14 | (39) | 1 | (3) |
| Pyrexia | 10 | (28) | 0 | |
| Diarrhea | 9 | (25) | 0 | |
| Nausea | 7 | (19) | 1 | (3) |
| Constipation | 6 | (17) | 0 | |
| Urinary tract infection | 6 | (17) | 3 | (8) |

*Includes 8 Phase lb patients.
¶1 of 4 occurred in Phase 1b.

The most commonly occurring treatment emergent adverse events (TEAEs) in greater than or at least 15% of patients of the 36 patients in Phase 1b and 2 is set forth in Table 5. No cases of hyperphosphatemia or ocular or nail toxicity were reported. The majority of TEAEs were predominantly grade 1, occurred early on study, and resolved on study treatment.

TABLE 4
Clinical Response - RECIST 1.1 Evaluable Population:

| | Total | WT | Mut/Fus |
|---|---|---|---|
| | (n = 22) | (n = 15) | (n = 7) |
| ORR*, n (%) | 9 (41) | 6 (40) | 3 (43) |
| Median follow-up time, mo | 9.4 | 12.0 | 8.7¶ |
| Disease control rate (DCR): | NR | NR | NR |
| partial response (PR)/stable | | | |
| disease (SD) (95% CI) | | | |

*1 patient withdrew consent prior to confirming PR and one patient had pseudoprogression at next scan.
¶Patients in mut/fus have approximately 4 months shorter follow-up.

The most commonly occurring vofatamab-related TEAEs in greater than or at least 15% of patients of the 36 patients in Phase 1b and 2 is set forth in Table 6. No grade 3 or greater vofatamab-related TEAEs were reported. The most common vofatamab-related TEAEs were fatigue and diarrhea.

TABLE 6
Vofatamab-related TEAEs in ≥15% of Patients in Phase 1b/2:

| Medical Dictionary for | | |
|---|---|---|
| Regulatory Activities, | Any Grade, n (%) | Grade ≥3, n (%) |
| Preferred Term | n = 36* | n = 36 |
| Fatigue | 8 (22) | 0 |
| Diarrhea | 6 (17) | 0 |

*Includes 8 Phase 1b patients.

The lead-in window of vofatamab was well tolerated, and paired biopsies were safely obtained from the majority of patients (80%). Combination therapy with pembrolizumab was also well tolerated. No dose reductions of vofatamab occurred and interruptions were rare with 6 interruptions over 303 cycles. Serious AEs occurring in ≥2 patients were urinary tract infection (n=2; 6%) and acute kidney injury (n=2; 6%). All serious SEs were unrelated to vofatamab.

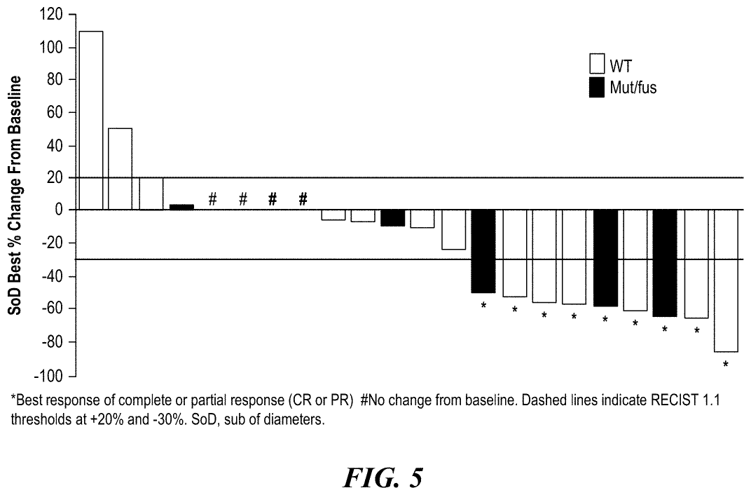

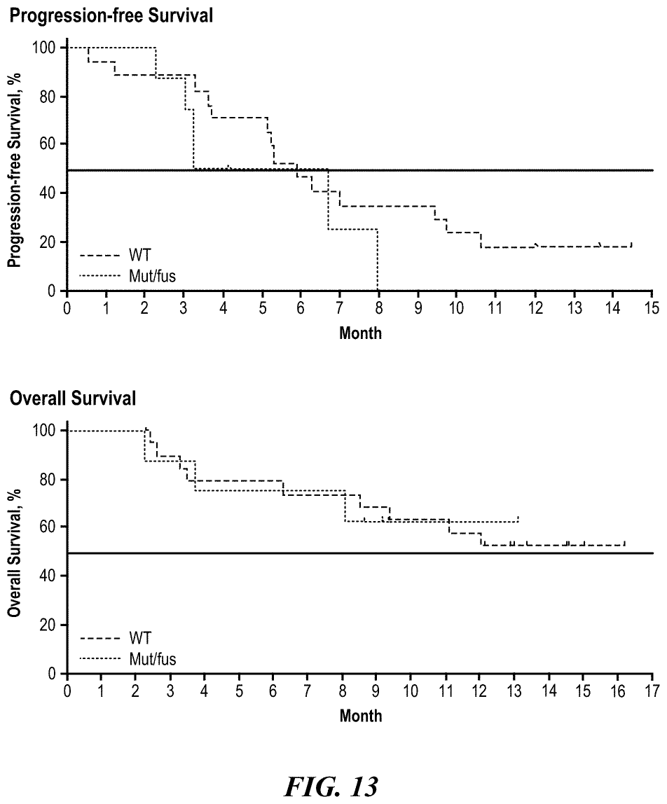

In the RECIST 1.1-evaluable Phase 2 study population, the ORR was approximately twice that of pembrolizumab alone. The ORR for all 22 patients with evaluable lesions was 40% (9 of 22). The ORR for WT was 40% (6 of 15) and for Mut/Fus was 43% (3 of 7). Thus, responses were similar regardless of FGFR3 Mut/Fus or WT status (FIGS. 5, 13). The progression-free survival (PFS) trend appears favorable for patients treated with combination therapy (5.9 months; 95% CI 3.6, 8.0) versus pembrolizumab alone (2.1 months; 95% CI 2.0, 2.2). The DOR is shorter than previously reported with CPIs alone, and doesn't seem to predict OS. The median OS has not been reached with all WT patients being followed for at least 12 mo follow-up.

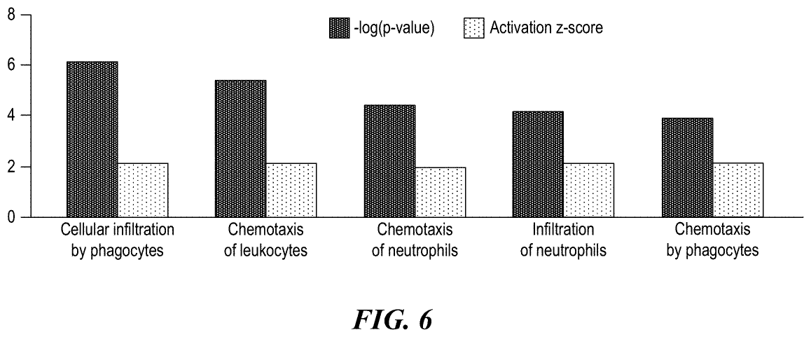

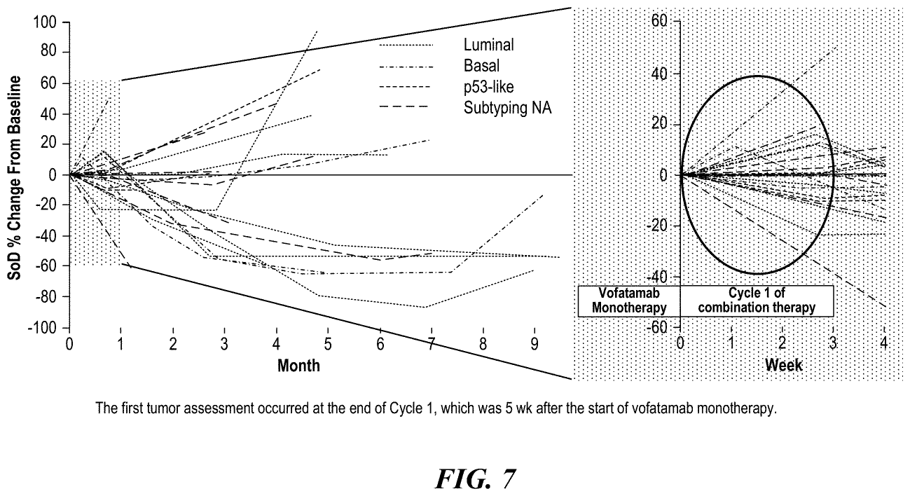

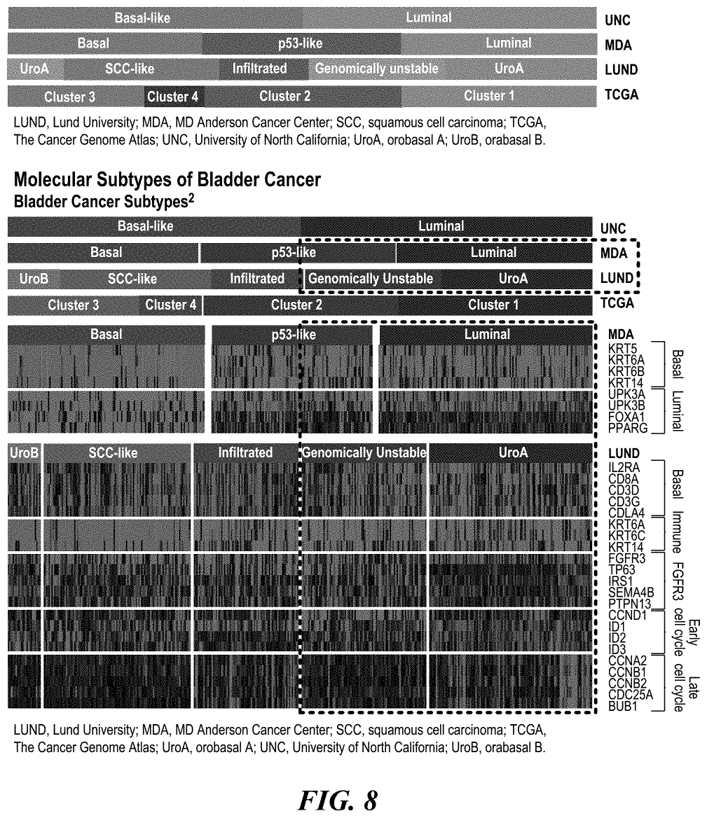

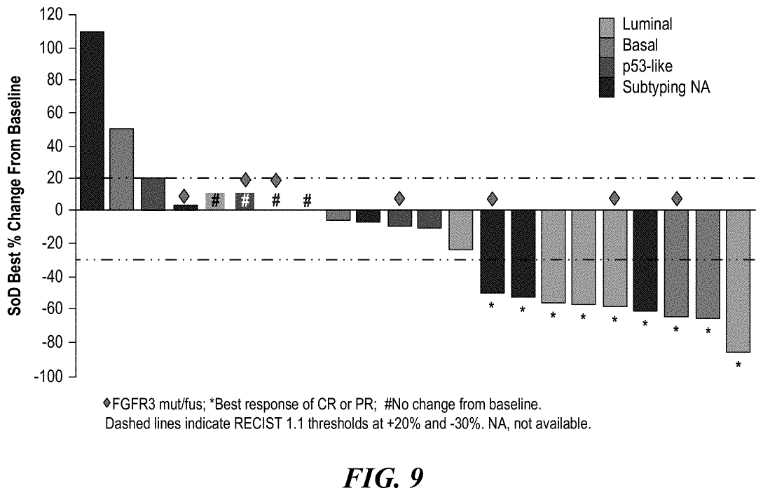

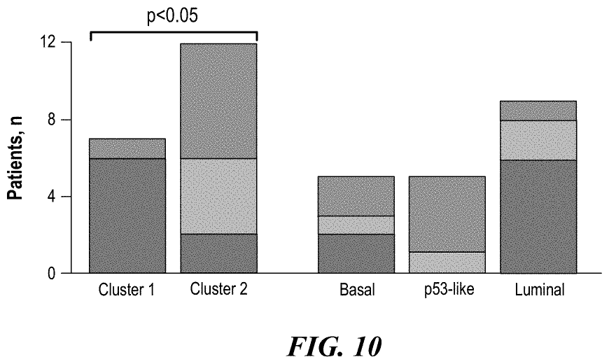

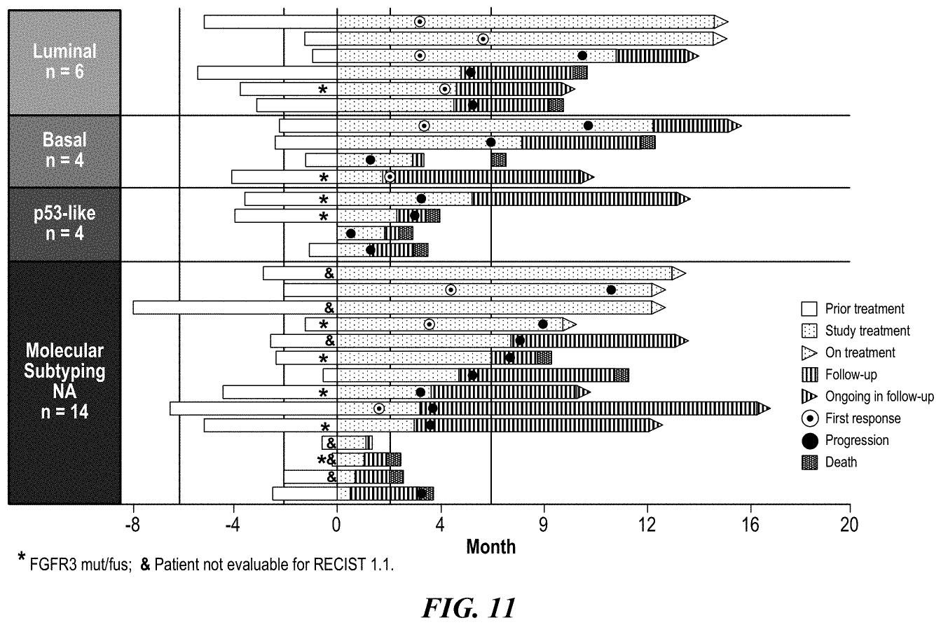

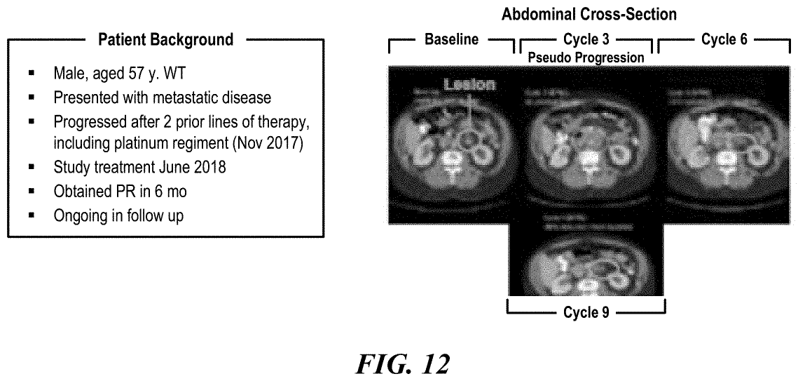

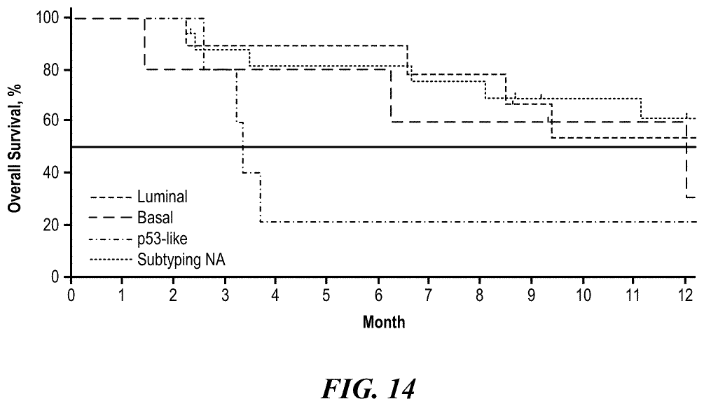

Lead-in vofatamab monotherapy induced upregulation of genes associated with inflammatory response and immune changes and immune pathway alterations in responders (FIG. 6). Clinical response correlated with molecular subgroups, and an increased response rate (6 of 8) was observed in the luminal subgroup (i.e., in subjects with tumors that were immunologically cold) (FIGS. 7-12), suggesting that combination therapy may be particularly effective for this subtype. The presence of cancer-associated fibroblasts (CAFs) appears to be a possible mechanism associated with lack of response. Patients with p53-like tumors appeared to respond poorly (FIG. 14).

Vofatamab results in immunological changes through FGFR3 inhibition. In a preliminary subtype study, vofatamab combined with pembrolizumab improves responses in bladder cancer subtypes, except the p53-like group. Resistance to treatment may be associated with p53-like subgroup of patients. Several patients derived continued clinical benefit from treatment beyond RECIST 1.1 progression.

### REFERENCES

1. American Cancer Society, Cancer Facts & FIGS. 2018)
2. Bai et al. Cancer Res 70(19):7630-7639 (Aug. 13, 2010)
3. Bellmunt et al. J Clin Oncol 27(27):4454-4461 (Aug. 17, 2009)
4. Brooks et al. Clin Cancer Res 18(7):1855-1862 (Mar. 2, 2012)
5. Bumbaca et al. MAbs 3(4):376-386 (Jul. 1, 2011)
6. Costantini et al. Scientific World Journal 11:1981-1994 (Oct. 26, 2011)
7. Denrauer et al. Proc Natl Acad Sci USA 111(8):3110-3115 (Feb. 11, 2014)
8. Eble et al. World Health Organization Classification of Tumors. Pathology and Genetics of Tumors of the Urinary System and Male Genital Organs. Lyon, France. IARC Press (2004)
9. Jemal et al. C A Cancer J Clin. 61(2):69-90 (March-April 2011)
10. Lafitte et al. Mol. Cancer 12:83 (Jul. 31, 2013)
11. Pardoll Nat Rev Cancer 12(4):252-264 (Mar. 22, 2012)
12. Siefker-Radtke et al. J Clin Oncol 20(5):1361-1367 (Mar. 1, 2002)
13. Sternberg et al. J Clin Oncol 19(10):2638-2646 (May 15, 2001)
14. Vallot et al. J Natl Cancer Inst 103(1):47-60 (Dec. 20, 2010)
15. von der Maase et al. J Clin Oncol 18(17):3068-3077 (September 2000)
16. Wu Nat Rev Cancer 5(9):713-725 (September 2005)
17. Zhao et al. Clin Cancer Res 16(23):5750-5758 (Dec. 1, 2010)

What is claimed is:

1. A method of treating luminal bladder cancer expressing wild-type FGFR3 in a subject in need thereof comprising administering to the subject a therapeutically effective amount of an FGFR3 inhibitor in combination with a therapeutically effective amount of a checkpoint inhibitor.

2. The method of claim 1, wherein the FGFR3 inhibitor is an antagonistic FGFR3 inhibitor.

3. The method of claim 2, wherein the antagonistic FGFR3 inhibitor is an antagonistic FGFR3 antibody.

4. The method of claim 3, wherein the antagonistic FGFR3 antibody comprises CDR-H1 comprising the amino acid sequence set forth in SEQ ID NO:1, CDR-H2 comprising the amino acid sequence set forth in SEQ ID NO:2, and CDR-H3 comprising the amino acid sequence set forth in SEQ ID NO:3.

5. The method of claim 4, wherein the antagonistic FGFR3 antibody comprises a heavy chain variable region comprising the amino acid sequence set forth in SEQ ID NO:7.

6. The method of claim 3, wherein the antagonistic FGFR3 antibody comprises CDR-L1 comprising the amino acid sequence set forth in SEQ ID NO:4, CDR-L2 comprising the amino acid sequence set forth in SEQ ID NO:5, and CDR-L3 comprising the amino acid sequence set forth in SEQ ID NO:6.

7. The method of claim 6, wherein the antagonistic FGFR3 antibody comprises a light chain variable region comprising the amino acid sequence set forth in SEQ ID NO:8.

8. The method of claim 1, wherein the FGFR3 inhibitor is vofatamab.

9. The method of claim 1, wherein the checkpoint inhibitor is a PD1 inhibitor.

10. The method of claim 9, wherein the PD1 inhibitor is an antagonistic PD-L1 antibody.

11. The method of claim 10, wherein the antagonistic PD-L1 antibody is selected from the group consisting of MEDI-4736, RG7446, BMS-936559, MSB0010718C, and MPDL3280A.

12. The method of claim 9, wherein the PD1 inhibitor is pembrolizumab.

13. A method of treating luminal bladder cancer expressing wild-type FGFR3 in a subject in need thereof comprising administering a therapeutically effective amount of an antagonistic FGFR3 inhibitor in combination with a therapeutically effective amount of a PD1 inhibitor.

14. The method of claim 13, wherein the antagonistic FGFR3 inhibitor is an antagonistic FGFR3 antibody.

15. The method of claim 14, wherein the antagonistic FGFR3 antibody comprises CDR-H1 comprising the amino acid sequence set forth in SEQ ID NO:1, CDR-H2 comprising the amino acid sequence set forth in SEQ ID NO:2, CDR-H3 comprising the amino acid sequence set forth in SEQ ID NO:3, and a heavy chain variable region comprising the amino acid sequence set forth in SEQ ID NO:7.

16. The method of claim 14, wherein the antagonistic FGFR3 antibody comprises CDR-L1 comprising the amino acid sequence set forth in SEQ ID NO:4, CDR-L2 comprising the amino acid sequence set forth in SEQ ID NO:5, CDR-L3 comprising the amino acid sequence set forth in SEQ ID NO:6, and a light chain variable region comprising the amino acid sequence set forth in SEQ ID NO:8.

17. The method of claim 13, wherein the FGFR3 inhibitor is vofatamab.

18. The method of claim 13, wherein the PD1 inhibitor is an antagonistic PD-L1 antibody.

19. The method of claim 18, wherein the antagonistic PD-L1 antibody is selected from the group consisting of MEDI-4736, RG7446, BMS-936559, MSB0010718C, and MPDL3280A.

20. The method of claim 13, wherein the PD1 inhibitor is pembrolizumab.

21. A method of treating a subject having cancer expressing wild-type FGFR3 in need thereof, the method comprising:
(a) screening the subject for a gene signature that correlates with one or more cancer-associated fibroblasts or for p53 expression;
(b) determining if the subject has the gene signature that correlates with the one or more cancer-associated fibroblasts or has p53 expression;
(c) based on the determining of step (b)—
(i) if the subject does not have the gene signature or p53 expression, administering a therapeutically effective amount of an FGFR3 inhibitor in combination with a therapeutically effective amount of a checkpoint inhibitor, and
(ii) if the subject does have the gene signature or p53 expression, administering a therapeutically effective amount of an FGFR3 inhibitor in combination with a therapeutically effective amount of a checkpoint inhibitor and an additional anti-cancer agent.

22. The method of claim 21, wherein the FGFR3 inhibitor is an antagonistic FGFR3 antibody.

23. The method of claim 21, wherein the antagonistic FGFR3 antibody comprises CDR-H1 comprising the amino acid sequence set forth in SEQ ID NO:1, CDR-H2 comprising the amino acid sequence set forth in SEQ ID NO:2, CDR-H3 comprising the amino acid sequence set forth in SEQ ID NO:3, and a heavy chain variable region comprising the amino acid sequence set forth in SEQ ID NO:7.

24. The method of claim 21, wherein the antagonistic FGFR3 antibody comprises CDR-L1 comprising the amino acid sequence set forth in SEQ ID NO:4, CDR-L2 comprising the amino acid sequence set forth in SEQ ID NO:5, CDR-L3 comprising the amino acid sequence set forth in SEQ ID NO:6, and a light chain variable region comprising the amino acid sequence set forth in SEQ ID NO:8.

25. The method of claim 21, wherein the FGFR3 inhibitor is vofatamab.

26. The method of claim 21, wherein the checkpoint inhibitor is a PD1 inhibitor.

27. The method of claim 26, wherein the PD1 inhibitor is an antagonistic PD-L1 antibody selected from the group consisting of MEDI-4736, RG7446, BMS-936559, MSB0010718C, and MPDL3280A.

28. The method of claim 21, wherein the PD1 inhibitor is pembrolizumab.

29. The method of claim 21, wherein the cancer is luminal bladder cancer.


SEQUENCE LISTING (10 sequences)

SEQ ID NO: 1 - protein, 10 aa, Artificial Sequence
  CDR-H1
GFTFTSTGIS

SEQ ID NO: 2 - protein, 18 aa, Artificial Sequence
  CDR-H2
GRIYPTSGSTNYADSVKG

SEQ ID NO: 3 - protein, 20 aa, Artificial Sequence
  CDR-H3
ARTYGIYDLYVDYTEYVMDY

SEQ ID NO: 4 - protein, 11 aa, Artificial Sequence
  CDR-L1
RASQDVDTSLA

SEQ ID NO: 5 - protein, 7 aa, Artificial Sequence
  CDR-L2
SASFLYS

SEQ ID NO: 6 - protein, 9 aa, Artificial Sequence
  CDR-L3
QQSTGHPQT

SEQ ID NO: 7 - protein, 123 aa, Artificial Sequence
  heavy chain variable region
EVQLVESGGGLVQPGGSLRLSCAASGFTFTSTGISWVRQAPGKGLEWVGRIYPTSGSTNYADSVKGRFTISADTSKNTAYLQMNSLRAEDTAVYYCARTYGIYDLYVDYTEYVMDYWGQGTLV

SEQ ID NO: 8 - protein, 108 aa, Artificial Sequence
  light chain variable region
DIQMTQSPSSLSASVGDRVTITCRASQDVDTSLAWYKQKPGKAPKLLIYSASFLYSGVPSRFSGSGSGTDFTLTISSLQPEDFATYYCQQSTGHPQTFGQGTKVEIKR

SEQ ID NO: 9 - protein, 457 aa, Artificial Sequence
  heavy chain human IgG1
EVQLVESGGGLVQPGGSLRLSCAASGFTFTSTGISWVRQAPGKGLEWVGRIYPTSGSTNYADSVKGRFTISADTSKNTAYLQMNSLRAEDTAVYYCARTYGIYDLYVDYTEYVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, Artificial Sequence
  light chain variable region + human Ig kappa chain C
DIQMTQSPSSLSASVGDRVTITCRASQDVDTSLAWYKQKPGKAPKLLIYSASFLYSGVPSRFSGSGSGTDFTLTISSLQPEDFATYYCQQSTGHPQTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC